(19) United States
(12) Patent Application Publication
(10) Pub. No.: US 2026/0224392 A1
(43) Pub. Date: Aug. 6, 2026

(54) CRYOGENIC BED

(71) Applicant: STARPOOL S.R.L., Ziano di Fiemme (TN) (IT)

(72) Inventor: Riccardo TURRI, Ziano di Fiemme (TN) (IT)

(21) Appl. No.: 19/160,283

(22) PCT Filed: Nov. 14, 2023
(86) PCT No.: PCT/IB2023/061471

§ 371 Date: Aug. 27, 2025

(30) Foreign Application Priority Data
Mar. 2, 2023 (IT) 102023000003795

(51) Int. Cl. A61F 7/00

(52) U.S. Cl. CPC A61F 7/0053; A61F 7/0097; A61F 2007/0056; A61F 2007/0063

(57) ABSTRACT

The cryogenic bed (1) comprises at least one base body (2) resting on the ground and provided with at least one housing compartment (3) of a flotation liquid and with at least one upper opening (4) for accessing to the housing compartment (3), wherein comprises cryogenic treatment means (8) of a user comprising at least one holding and covering element (9) associated with the upper opening (4) and adapted to hold the user in a lying position, the holding and covering element (9) being provided with at least one refrigeration flap (10) which can be wrapped around at least one portion of the body of the user to perform a cryogenic treatment on the latter.

### TECHNICAL FIELD

The present invention relates to a cryogenic bed.

### BACKGROUND ART

To date, the practice of performing beauty treatments of the thermal type, i.e., exposing the body of a patient to either a heating agent or a cooling agent, possibly even in combination thereof, is well known and increasingly popular.

Within the scope of this disclosure, reference is mainly made to thermal treatments of the body involving the use of a cooling agent and, specifically, to cryogenic treatments.

Cryotherapy, also known as cold therapy, is based on the therapeutic properties of cold and is used in both medical field and purely beauty field.

In the medical field, cryotherapy is used to treat, e.g., contractures or injuries to the musculoskeletal system or diseases affecting the skin.

In the beauty field, cryogenic treatments are widely used because of the beneficial effects they bring to users from the psychophysical point of view and improvement in the level of toning of muscles and/or skin.

Among the many beneficial effects of cryogenic treatments are the slowing of skin aging and improved control of weight and water retention.

Usually, cryogenic beauty treatments involve fully or partly immersing the body of a user in a tub containing a cooling element such as, e.g., ice, in the form of cubes and/or ice water.

Alternatively or in combination thereof, the cooling element can be composed of liquid nitrogen which is sprayed onto the body of the person being treated.

Performing cryogenic treatments in the manner just mentioned, however, has some drawbacks which are mainly related to the difficulties met during their execution.

Particularly, in order to be able to perform cryogenic treatments optimally, it is necessary to implement a series of procedures which are ancillary to the treatment itself which are implemented both before and after the actual treatment.

First of all, the user who wants to undergo cryogenic treatment must undress to avoid wetting clothes once immersed in the refrigerant liquid or fluid.

Following the treatment, because the skin of the treated user is wet, it is necessary for the skin to be dried and then allow the user to dress.

It is easy to appreciate how the implementation of such procedures ancillary to the treatment is not quick and easy.

Such conditions may even discourage users from undergoing cryogenic treatments of the known type, especially when they have little time to spend for the treatment.

In this regard, it is necessary to emphasize that in the case where cryogenic treatment is used by a sportsman to de-contract and/or relax muscles following physical effort, it is desirable to perform the treatment immediately after the physical effort is performed to maximize the beneficial effects of cold on the sportsman's body.

The fact of having to prepare for treatment by undressing lengthens the time between the physical activity and immersion in the tub, reducing the beneficial effects of cold on the body and consequently discouraging athletes from undergoing such treatments.

Additionally, not all users like direct skin contact with a refrigerant liquid or gas, not to mention the fact that this practice can be dangerous to the safety of the user.

### DESCRIPTION OF THE INVENTION

The main aim of the present invention is to devise a cryogenic bed which allows promoting cryogenic treatments by simplifying and speeding up their performance.

Within the scope of the present aim, the present invention aims to devise a cryogenic bed which allows decreasing the number and/or duration of any practices which are preparatory to the treatment itself or to be performed at the end of the same.

Another object of the present invention is to devise a cryogenic bed which allows meeting the needs of users, especially in terms of saving time for the same psychophysical benefits obtained as a result of treatment.

As part of this aim, one object of the present invention is to devise a cryogenic bed which allows the user to devote him/herself completely to the treatment, without having to undergo other practices such as undressing and dressing, or without having a refrigerant fluid directly contacting with their skin.

Still, a further object of the present invention is to devise a cryogenic bed which makes cryogenic treatments particularly appealing to different types of users, such as, e.g., athletes, who need to quickly treat any contractures and/or musculoskeletal fatigue derived from the performance of physical exercise, or users who have little time to devote to treatment.

Another object of the present invention is to devise a cryogenic bed which allows the aforementioned drawbacks of the prior art to be overcome within the framework of a simple, rational, easy and effective to use as well as affordable solution.

The above objects are achieved by this cryogenic bed having the characteristics of claim 1.

### BRIEF DESCRIPTION OF THE DRAWINGS

Other characteristics and advantages of the present invention will become more apparent from the description of a preferred, but not exclusive, embodiment of a cryogenic bed, illustrated by way of an indicative, yet non-limiting example, in the accompanying tables of drawings in which:

FIG. 1 is an axonometric view of a cryogenic bed according to the invention;

FIG. 2 is an exploded axonometric view of the cryogenic bed according to the invention.

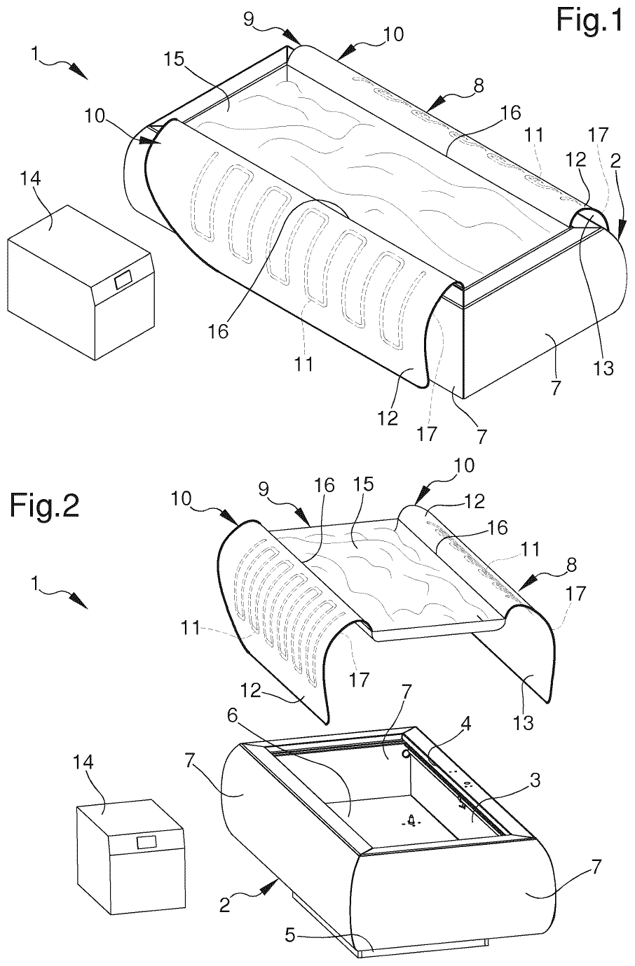

### EMBODIMENTS OF THE INVENTION

With particular reference to these figures, reference numeral 1 globally denotes a cryogenic bed.

The bed 1 comprises at least one base body 2 resting on the ground and provided with at least one housing compartment 3 of a flotation liquid and with at least one upper opening 4 for accessing the housing compartment 3.

The base body 2 comprises a resting element 5 having a substantially parallelepiped conformation and arranged resting onto the ground.

The base body 2 comprises at least one containment element 6, 7 associated above the resting element 5, which is substantially hollow and internally defining the housing compartment 3.

Specifically, the containment element 6, 7 comprises a substantially flat bottom wall 6, associated with the resting element 5 and arranged substantially horizontally, and a plurality of side walls 7 associated with the bottom wall 6 and arranged substantially orthogonal to the latter.

The side walls 7 define the upper opening 4 at the top.

The upper opening 4 has a substantially rectangular conformation.

Preferably, the flotation liquid is water.

According to the invention, the bed 1 comprises cryogenic treatment means 8 of a user comprising at least one holding and covering element 9 associated with the upper opening 4 and adapted to hold the user in a lying position.

The holding and covering element 9 is provided with at least one refrigeration flap 10 which can be wrapped around at least one portion of the body of the user to perform a cryogenic treatment on the latter.

The holding and covering element 9 is arranged at least partly resting on the edge of the upper opening 4 and partly resting on the flotation liquid.

Conveniently, the holding and covering element 9 is associated with the upper opening 4 by interposition of joining means.

Preferably, the joining means comprise a frame-like element associated with the holding and covering element 9 and couplable to size with the upper opening 4. In detail, the frame-like element and the upper opening 4 define an interlocking coupling.

Preferably, the holding and covering element 9 is made at least partly of plastic material.

Conveniently, the plastic material for making the holding and covering element 9 is flexible and waterproof to the flotation liquid.

While using the bed 1, the user lies on the holding and covering element 9 and floats on the flotation fluid.

At the same time, the user arranges the refrigeration flap 10 around at least one portion of his or her body in such a way as to cover it; this allows the latter to benefit evenly from the cryogenic treatment.

The special technical expedient of providing the bed 1 with the holding and covering element 9 allows users to undergo cryogenic treatment without their skin coming into direct contact with a refrigerant fluid.

In this way, users do not need to undress before undergoing the cryogenic treatment and do not even have to dry their skin once the treatment is over.

Again, the fact of placing the holding and covering element 9 floating on the flotation liquid allows the user of the bed 1 to benefit from the relaxation effect generated by floating while lying on a liquid.

Advantageously, the refrigeration flap 10 comprises at least one cooling coil 11 which can be run through by at least one refrigerant fluid and developing on at least one portion of the refrigeration flap 10.

The body of the user placed in contact with the refrigeration flap 10 is cooled by the refrigerant fluid running through the cooling coil 11.

Specifically, the refrigerant fluid, as will be described in detail below, is at a predefined temperature which is lower than the temperature of the user's body and, when it comes into contact with the latter, it heats up by drawing heat from the user's body, which is thus chilled and subjected to cryogenic treatment.

Advantageously, the cooling coil 11 has a direction of longitudinal development which is substantially parallel to the user's body.

Preferably, in use, the cooling coil 11 lies on a substantially horizontal plane.

With particular reference to the embodiment shown in the figures, the cooling coil 11 spans almost the entire surface of the refrigeration flap 10 so that it contacts substantially the entire body of the user.

In detail, the cooling coil 11 is not arranged where the portion of the refrigeration flap 10 is located which, in use, can be positioned in contact with the user's head. In this way, direct contact between the refrigeration flap 10 and the user's head, which is known to be more delicate and more sensitive to temperature changes, can be avoided.

Advantageously, the refrigeration flap 10 comprises at least one containment compartment 17 adapted to contain the cooling coil 11.

In other words, the cooling coil 11 is not directly accessible from the outside.

The fact of arranging the cooling coil 11 in the containment compartment 17 prevents the user's body from directly contacting the cooling coil 11, thus improving the comfort level perceived by the user him/herself.

In fact, the cooling coil 11 is, usually, of the type of a tubular body made of plastic material, the direct contact of which with the user's body may be unpleasant.

Therefore, in order not to create discomfort upon contact with the user's body, the cooling coil 11 is placed inside the containment compartment 17.

For example, the plastic material for making the cooling coil 11 is polyvinyl chloride (PVC) or polyurethane (PU).

In addition, the fact of placing the cooling coil 11 inside the containment compartment 17 protects the latter from any external physical agents that may damage it.

Preferably, the refrigeration flap 10 comprises at least a first sheet element 12 and at least a second sheet element 13 mutually coupled to define the containment compartment 17.

In actual facts, the edges of the sheet elements 12, 13 are associated with each other so that the sheet elements themselves internally define an empty volume in which the cooling coil 11 is housed.

For example, the edges of the sheet elements 12, 13 are heat-sealed to each other. Advantageously, the bed 1 comprises heat treatment means 14 adapted to cool the refrigerant fluid and to send it to the cooling coil 11 at the predefined temperature.

In accordance with a preferred embodiment of the bed 1, the refrigerant fluid is in a liquid state.

In the present case, the refrigerant fluid is water.

It cannot, however, be ruled out from the scope of this disclosure that the refrigerant fluid may be in the gaseous state.

The predefined temperature is a temperature value considered optimal in order to achieve the greatest benefits on the user's body when carrying out the cryogenic treatment.

Advantageously, the value of the predefined temperature may vary depending on the result to be achieved and/or in relation to the user's preferences.

In detail, the predefined temperature is of between −10° C. and 10° C., preferably of between −5° C. and 8° C., even more preferably of between 4° C. and 6° C.

Generally, the predefined temperature to which the refrigerant fluid is brought is of between 4° C. and 6° C.; it is, in fact, possible to note that the exposure of the user's body to a temperature within this range provides satisfactory comfort levels and excellent results in terms of the quality of the cryogenic treatment.

Preferably, the heat treatment means 14 comprise a treatment device of the refrigerant fluid which performs a refrigeration cycle on the refrigerant fluid in order to bring it to a predetermined temperature.

The heat treatment means 14 are connected to the cooling coil 11 in a fluid-operated manner and preferably comprise a pumping device adapted to move the refrigerant fluid from the treatment device to the cooling coil 11.

With particular reference to the embodiment shown in the figures, the heat treatment means 14 are arranged outside the base body 2.

In alternative embodiments not shown in the figures, the heat treatment means 14 are built in the base body 2.

Conveniently, the bed 1 comprises at least one management and control unit configured to enable the monitoring of the heat treatment means 14 and the maintenance of the refrigerant fluid at the predefined temperature.

Preferably, the heat treatment means 14 comprise a temperature sensor (not shown in the figures) adapted to detect the temperature of the refrigerant fluid.

The management and control unit (not shown in the figures) is operationally connected to the temperature sensor, which is adapted to send the detected temperature value to the management and control unit.

In more detail, the management and control unit is programmable with the predefined temperature value and is operationally configured to compare the temperature value detected by the temperature sensor with the predefined temperature value.

In the event of the management and control unit detecting a difference between the detected temperature value and the predefined temperature value, the heat treatment means 14 are activated so as to change the detected temperature value to be substantially coincident with the predefined temperature value.

In this way, the temperature of the refrigerant fluid sent to the cooling coil 11 can be controlled so that the refrigeration flap 10 is at the predefined temperature thus allowing the efficiency of cryogenic treatment to be ensured.

Conveniently, the holding and covering element 9 comprises at least two refrigeration flaps 10 arranged, in use, mutually opposite with respect to the body of the user and adapted to wrap it by overlapping each other.

With particular reference to the embodiment shown in the figures, the holding and covering element 9 comprises two refrigeration flaps 10.

Precisely, the holding and covering element 9 comprises a central portion 15 located between the refrigeration flaps 10 and arranged to rest on the free surface of the flotation liquid through the upper opening 4.

The central portion 15 is adapted to hold the body of the user from below.

The central portion 15 has a substantially rectangular conformation and is developed longitudinally along an axis of longitudinal development which is substantially parallel to the bottom wall 6.

The central portion 15 has a surface extension which is substantially coincident with the area of the upper opening 4.

The refrigeration flaps 10 are arranged opposite with respect to a median plane passing through the holding and covering element 9.

In more detail, the edges of the refrigeration flaps 10 are associated with the central portion 15; this means that the refrigeration flaps 10 are associated with two sides 16 of the central portion 15, arranged opposite each other and developing substantially parallel to the axis of longitudinal development of the central portion 15.

Conveniently, the refrigeration flaps 10 develop substantially along the entire length of the sides 16.

Preferably, during the use of the bed 1, the user lies on the central portion 15 so that the longitudinal axis of the body of the user him/herself is substantially coincident with the axis of longitudinal development of the central portion 15 and arranges the refrigeration flaps 10 above his or her own body by overlapping them, wrapping the body within the holding and covering element 9.

The special expedient of providing two refrigeration flaps 10, each provided with a respective cooling coil 11, makes it possible to achieve a synergistic combination of thermal parameters ensuring a physiological response and benefits quite similar to those obtainable during the performance of cryogenic treatments of known type, i.e., in which immersion and direct contact with a refrigerant fluid are provided.

In detail, these thermal parameters consist of the heat transfer coefficient between the user's body and the refrigeration flaps 10, of the immersion coefficient of the user's body and of the percentage of contact between the user's skin and the refrigeration flaps 10; the aforementioned combination of parameters makes it possible to ensure a satisfactory body sensation comparable to well-known cryogenic immersion treatments.

In addition, the fact of having two cooling coils 11 allows for an improved level of heat exchange between the user's body and the refrigerant fluid; in fact, recirculation of the refrigerant fluid within the cooling coil 11 ensures that a constant operating temperature is maintained during the treatment session on the body surface.

Again, the presence of the refrigeration flaps 10 and of the relevant cooling coils 11 resting on the user's body increases the overall weight of the mass resting on the holding and covering element 9 compared to the case where these refrigeration flaps 10 are not present.

In this way, the user's degree of immersion in the flotation fluid can be increased, thus improving the sense of well-being perceived by the same user.

In addition, under such conditions, the degree of adhesion can be improved of the holding and covering element 9 to the user's body which is determined by the hydrostatic response of the flotation liquid.

The fact of providing two refrigeration flaps 10 allows the effects of the cryogenic treatment to be maximized.

Preferably, the flotation liquid has the predefined temperature.

In other words, the flotation liquid has a temperature of between −10° C. and 10° C., preferably of between −5° C. and 8° C., even more preferably of between 4° C. and 6° C.

The fact of bringing the flotation liquid to the predefined temperature also allows the central portion 15 of the holding and covering element 9 to be refrigerated, so that the portion of the user's body resting on the same central portion 15 can be directly refrigerated.

In actual facts, the entire holding and covering element 9 is refrigerated and the user is completely wrapped within a refrigerated body so as to increase the effects and benefits of performing the cryogenic treatment.

Conveniently, the bed 1 comprises refrigeration means adapted to cool the refrigerant liquid to the predefined temperature.

According to a preferred embodiment of the bed 1, the refrigeration means coincide with the heat treatment means 14.

In this case, the treatment device performs a refrigeration cycle on the flotation fluid in order to bring it to the predefined temperature and the pumping device moves the refrigerated flotation liquid from the treatment device itself to the housing compartment 3.

According to an alternative embodiment of the bed 1, the refrigeration means are separate from the heat treatment means 14 and comprise a refrigeration device which is adapted to perform a refrigeration cycle on the flotation liquid and a pump adapted to move the refrigerated flotation liquid from the refrigeration device towards the housing compartment 3.

The management and control unit is operationally connected to the refrigeration means and regulates the operation thereof, similarly to the regulation of the heat treatment means 14, so that the flotation liquid sent to the housing compartment 3 is maintained at the predefined temperature.

The operation of the bed 1 according to the invention is as follows.

Initially, the heat treatment means 14 and the refrigeration means are activated in order to bring the refrigerant fluid and the flotation fluid to the predefined temperature.

At the same time, the management and control unit compares the detected temperature of the refrigerant fluid and of the flotation liquid with the predefined temperature and takes action on the heat treatment means 14 and/or on the refrigeration means in order to make these values substantially coincident.

The user positions him/herself on the holding and covering element 9 and wraps the refrigeration flaps 10 around his/her own body.

It has in practice been ascertained that the described invention achieves the intended objects.

First of all, the use of the holding and covering element provided with two refrigeration flaps makes it possible to speed up and simplify the execution of the cryogenic treatments, since it reduces the frequency and duration of any practices which are preparatory to the treatment or to be carried out at its conclusion.

In fact, due to the presence of the holding and covering element, users undergo cryogenic treatment without their skin coming into direct contact with the refrigerant fluid.

In this way, users do not need to undress before undergoing the cryogenic treatment and do not even have to dry their skin once the treatment is over.

This also results in a significant reduction in the overall time a user must spend to undergo cryotherapy treatments.

In this way, the bed according to the invention allows making the cryogenic treatments desirable to a wide range of users comprising athletes, who need to quickly treat any contractures and/or musculoskeletal fatigue derived from the performance of physical exercise.

Additionally, the fact of placing the holding and covering element resting on the flotation liquid allows the bed's user to benefit from the relaxation effect generated by floating while lying on a liquid.

Again, the use of the bed according to the invention allows for the nullification of any risks to the user from immersion of the body in cold water and/or exposure to a cryogenic gas, such as liquid nitrogen.

1. Cryogenic bed (1), comprising at least one base body (2) resting on the ground and provided with at least one housing compartment (3) of a flotation liquid and with at least one upper opening (4) for accessing to said housing compartment (3), characterized by the fact that it comprises cryogenic treatment means (8) of a user comprising at least one holding and covering element (9) associated with said upper opening (4) and adapted to hold said user in a lying position, said holding and covering element (9) being provided with at least one refrigeration flap (10) which can be wrapped around at least one portion of the body of said user to perform a cryogenic treatment on the latter.

2. Bed (1) according to claim 1, characterized by the fact that said at least one refrigeration flap (10) comprises at least one cooling coil (11) which can be run through by at least one refrigerant fluid and developing on at least one portion of said refrigeration flap (10).

3. Bed (1) according to claim 1, characterized by the fact that said at least one refrigeration flap (10) comprises at least one containment compartment (17) adapted to contain said cooling coil (11).

4. Bed (1) according to claim 3, characterized by the fact that said at least one refrigeration flap (10) comprises at least a first sheet element (12) and at least a second sheet element (13) mutually coupled to define said containment compartment (17).

5. Bed (1) according to claim 1, characterized by the fact that it comprises heat treatment means (14) adapted to cool said refrigerant fluid and to send it to said cooling coil (11) at a predefined temperature.

6. Bed (1) according to claim 5, characterized by the fact that said heat treatment means (14) are arranged outside said base body (2).

7. Bed (1) according to claim 5, characterized by the fact that said heat treatment means (14) are built in said base body (2).

8. Bed (1) according to claim 1, characterized by the fact that it comprises at least one management and control unit configured to enable the monitoring of said heat treatment means (14) and the maintenance of said refrigerant fluid at said predefined temperature.

9. Bed (1) according to claim 8, characterized by the fact that said predefined temperature is comprised between −10° C. and 10° C., preferably between −5° C. and 8° C., even more preferably between 4° C. and 6° C.

10. Bed (1) according to one claim 1, characterized by the fact that said holding and covering element (9) comprises at least two of said refrigeration flaps (10) arranged, in use, mutually opposite relative to the body of said user and adapted to wrap it by overlapping each other.

11. Bed (1) according to claim 1, characterized by the fact that said holding and covering element (9) comprises a central portion (15) located between said refrigeration flaps (10) and arranged to rest on the free surface of said flotation liquid through said upper opening said central portion (15) being adapted to hold the body of said user from below.

12. Bed (1) according to claim 1, characterized by the fact that said flotation liquid has said predefined temperature.
